(19) United States
(12) Patent Application Publication
(10) Pub. No.: US 2010/0221365 A1
(43) Pub. Date: Sep. 2, 2010

(54) USE OF GINSENG FRACTIONS FOR PREVENTING OR REDUCING HEMATOLOGICAL MALIGNANCIES

(75) Inventors: Sandra C. Miller, Montreal (CA); Jacqueline Shan, Edmonton (CA)

Correspondence Address: ROTHWELL, FIGG, ERNST & MANBECK, P.C., 1425 K STREET, N.W., SUITE 800, WASHINGTON, DC, 20005, US

(73) Assignee: AFEXA LIFE SCIENCES INC., Edmonton (CA)

(21) Appl. No.: 12/394,313

(22) Filed: Feb. 27, 2009

(51) Int. Cl. A61K 36/258; A61P 7/00; A61P 35/02

(52) U.S. Cl. 424/728

(57) ABSTRACT

The invention is directed to ginseng fractions, methods of preventing or reducing hematological malignancies such as leukemia, and methods of elevating NK cells in a subject by administering to the subject an effective amount of a ginseng fraction, a pharmaceutical composition comprising the fraction in combination with another medicament or with one or more pharmaceutically acceptable carriers including food items. The fraction may be made from Panax quinquefolius or may be selected from CVT-E002, PQ₂, PQ₂₂₃ and purified fractions from CVT-E002, PQ₂ and PQ₂₂₃.

### FIELD OF THE INVENTION

This invention relates to ginseng fractions, methods of preventing or reducing a hematological malignancy such as leukemia, and methods of elevating NK cells in a subject by administering to the subject an effective amount of the ginseng fraction, or a pharmaceutical composition or food item comprising the ginseng fraction.

### BACKGROUND OF THE INVENTION

For hundreds of years, the use of certain non-toxic agents such as herbal compounds has been widely accepted for a variety of physiological conditions, especially in the Orient. Panax ginseng C. A. Meyer is the best known traditional Chinese medicine. The important pharmacological activities of ginseng extracts, alone or in combination with other drugs, include alleviation of renal impairment, prevention of stress, modulation of immunological responsiveness and inhibition of carcinogenesis. American ginseng, Panax quinquefolius, is another species of ginseng which has gained popularity as a health supplement having many beneficial health effects.

There have been several attempts in the prior art to isolate and elucidate the structure of various components present in ginseng to test for the effectiveness of these compounds in treating hematological malignancies; see for example, Fujimoto et al., in Chem Pharm Bull (Tokyo), 39(2):521-3 (1991); Yi et al, in Zhongguo ZhongXi Yi Jie He Za Zhi, 13(12):722-4, 708 (1993); Hasegawa et al., in Planta Med., 61 (5):409-13 (1995); Gao et al., in Zhongguo Zhong Xi Yi Jie He Za Zhi, 19(1 ):17-9 (1999); Keum et al., in Cancer Lett, 13;150(1):41-8 (2000) and Mutat. Res., 523-24:75-85 (2003); Wang et al., in Zhongguo ZhongXi Yi Jie He Za Zhi, 14(6):1089-95 (2006); Chui et al., in Oncol Rep. 16(6): 1313-6 (2006). However, all of the aforementioned studies have been limited to testing the effectiveness of saponin (ginsenoside) fractions of the ginseng plant under in vitro conditions in treating hematological malignancies, rather than in preventing such diseases under in vivo conditions.

Nearly 44,000 people are diagnosed with leukemia every year in the United States, with an average of 22,000 deaths every year. Children may be affected by either acute (rapidly developing) and chronic (slowly developing) forms of leukemia. In children, about 98% of leukemias are acute. Acute childhood leukemias are further divided into acute lymphocytic leukemia (ALL) and acute myelogenous leukemia (AML), depending on whether lymphyocytes or myelocytes are involved. Approximately 60% of children with leukemia have ALL, and about 38% have AML. Although slow-growing chronic myelogenous leukemia may also be seen in children, it is very rare, accounting for fewer than 50 cases of childhood leukemia each year in the United States.

Both humoral and cell-mediated immune responses are deficient in infant mice (Mosier and Johnson, 1975; Spear and Edelman, 1974; Sherwin and Rowlands, 1975). B cells, T cells and macrophages are produced, but are functionally inactive (Spear et al., 1973; Velardi and Cooper, 1984; Holan et al., 1991; Chiscon et al., 1972; Klinman, 1976; Argyris, 1968). The lack of functional capacity of immune cells in infant mice may result from the presence of suppressor cells and/or suppressor factors (Landahl, 1976). The deficiency in immunological function of the infants of both mice and humans predisposes them to the development of a variety of diseases and assorted pathogens, including tumors of hemopoietic and immune cell origin, i.e., leukemias and lymphomas.

Spontaneous cytolytic activity, directed against assorted tumor cells, and virus-infected cells is mediated by non-B, non-T lymphoid lineage cells (lymphocytes). Cells mediating such spontaneous cytolytic activity are known as natural killer (NK) cells (Strober, 1984; Argyris, 1978, Maier et al., 1989; Calkins and Stutman, 1978; Hertell-Wulff and Strober, 1988; Argyris, 1981; Keissling et al., 1975; Biron and Welsh, 1982; Herberman et al., 1975; Riccardi et al., 1981). These cells have been recognized for decades as the “first line of defense” in tumor combat. However, mice under the age of three weeks show little or no NK cell-mediated function (Argyris, 1981; Lala et al., 1985; Miller et al., 1981), paralleling the deficiencies in other cell lineages in the immune system. In humans, such immaturity of the infant/juvenile NK cell-mediated immunity may be more than coincidentally concomitant with the relatively high frequency of leukemias and lymphomas in the pediatric population. In infant mice, the mechanism for the lack of NK cell-mediated functional activity results from active suppression of newly developing NK cells, since it is possible to remove infant-source NK cells from their suppressive environment (bone marrow, spleen), and demonstrate that they are as effective in tumor combat as are NK cells from adult sources (Dussault and Miller, 1995). NK cells in the infant are capable of responding (like adults) to the stimulating cytokine, IL-2, by proliferating and increasing their numbers, even though in situ, in vivo, their tumor cytolytic function is suppressed (Dussault and Miller, 1995). This functional capacity, however, is imbued upon them, almost instantly, at the time of weaning.

An effective method or vaccine to prevent or reduce hematological malignancies, such as leukemia and lymphoma, is desired. Such a method or vaccine should be able to effectively obstruct, delay or diminish the hematological malignancy, yet still be tolerable to subjects including children. There is a need for a natural, herbal fraction or composition which specifically displays such activities without causing deleterious side effects or discomfort. Western physicians have been reluctant to prescribe herbal medicines due to lack of scientific research of their preventative and therapeutic properties. However, herbal medicines do not require the lengthy development time and high costs normally encountered with synthetic drugs. Further, they are readily available and offer the subject a more comfortable and affordable alternative with minimal side effects compared to prescription medication or vaccines.

### SUMMARY OF THE INVENTION

In one embodiment, the present invention is directed to a fraction of ginseng for the prevention or reduction of hematological malignancies. In another embodiment, the present invention is directed to a fraction of ginseng for the elevation of NK cells. In another embodiment, the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng. In another embodiment, the fraction is a fraction of Panax quinquefolius. In another embodiment, the fraction is selected from the group consisting of CVT-E002, PQ₂, PQ₂₂₃ and purified fractions from CVT-E002, PQ₂ and PQ₂₂₃. In a further embodiment, the fraction is CVT-E002.

In another embodiment, the present invention is directed to a pharmaceutical composition comprising a fraction of ginseng in combination with another medicament or with one or more pharmaceutically acceptable carriers for the prevention or reduction of hematological malignancies. In another embodiment, the present invention is directed to a pharmaceutical composition comprising a fraction of ginseng in combination with another medicament or with one or more pharmaceutically acceptable carriers for the elevation of NK cells. In another embodiment, the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng. In another embodiment, the fraction is a fraction of Panax quinquefolius. In another embodiment, the fraction is selected from the group consisting of CVT-E002, PQ₂, PQ₂₂₃ and purified fractions from CVT-E002, PQ₂ and PQ₂₂₃. In a further embodiment, the fraction is CVT-E002.

In another embodiment, the present invention is directed to a food item comprising a ginseng fraction for the prevention or reduction of hematological malignancies. In another embodiment, the present invention is directed to a food item comprising a ginseng fraction for the elevation of NK cells. In another embodiment, the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng. In another embodiment, the fraction is a fraction of Panax quinquefolius. In another embodiment, the fraction is selected from the group consisting of CVT-E002, PQ₂, PQ₂₂₃ and purified fractions from CVT-E002, PQ₂ and PQ₂₂₃. In a further embodiment, the fraction is CVT-E002.

In another embodiment, the present invention is directed to use of a ginseng fraction for the preparation of a pharmaceutical composition or a food item for the prevention or reduction of hematological malignancies. In another embodiment, the present invention is directed to use of a ginseng fraction for the preparation of a pharmaceutical composition or a food item for the elevation of NK cells. In another embodiment, the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng. In another embodiment, the fraction is a fraction of Panax quinquefolius. In another embodiment, the fraction is selected from the group consisting of CVT-E002, PQ₂, PQ₂₂₃ and purified fractions from CVT-E002, PQ₂ and PQ₂₂₃. In a further embodiment, the fraction is CVT-E002.

In another embodiment, the present invention is directed to a method of preventing or reducing hematological malignancies comprising administering to a subject, when the subject is an infant, an effective amount of a ginseng fraction, a pharmaceutical composition comprising the fraction in combination with another medicament or with one or more pharmaceutically acceptable carriers including food items. In another embodiment, the hematological malignancy is an abnormal proliferation of blood cells, a disease of the lymph nodes or multiple myeloma. In another embodiment, the blood cells are leukocytes and/or erythrocyte precursors. In another embodiment, the abnormal proliferation of blood cells is selected from the group consisting of acute lymphocytic leukemia, acute myelogenous leukemia, chronic lymphocytic leukemia including the subtype hairy cell leukemia, and chronic myelogenous leukemia. In another embodiment, the acute myelogenous leukemia is erythroleukemia. In another embodiment, the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng. In another embodiment, the fraction is a fraction of Panax quinquefolius. In another embodiment, the fraction is selected from the group consisting of CVT-E002, PQ₂, PQ₂₂₃ and purified fractions from CVT-E002, PQ₂ and PQ₂₂₃. In another embodiment, the fraction is CVT-E002.

In yet another embodiment, the present invention is directed to a method of preventing leukemia in a subject comprising administering to a subject an effective amount of a ginseng fraction, a pharmaceutical composition comprising the fraction in combination with another medicament or with one or more pharmaceutically acceptable carriers including food items. In another embodiment, the fraction is made from a ginseng selected from the group consisting of Panax quinquefolius, Panax trifolia, Panax ginseng, Panaxjaponicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng. In another embodiment, the fraction is a fraction of Panax quinquefolius. In another embodiment, the fraction is selected from the group consisting of CVT-E002, PQ₂, PQ₂₂₃ and purified fractions from CVT-E002, PQ₂ and PQ₂₂₃. In another embodiment, the fraction is CVT-E002. In a further embodiment, the subject is a child.

In yet another embodiment, the present invention is directed to a method of producing elevated NK cells in an adult subject comprising administering to the subject when the subject is an infant an effective amount of a ginseng fraction. In another embodiment, the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng. In another embodiment, the fraction is a fraction of Panax quinquefolius. In another embodiment, the fraction is selected from the group consisting of CVT-E002, PQ₂, PQ₂₂₃ and purified fractions from CVT-E002, PQ₂ and PQ₂₂₃. In another embodiment, the fraction is CVT-E002.

In yet another embodiment, the present invention is directed to a method of increasing NK cells in an infant, comprising administering to an infant an effective amount of a ginseng fraction. In another embodiment, the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng. In another embodiment, the fraction is a fraction of Panax quinquefolius. In another embodiment, the fraction is selected from the group consisting of CVT-E002, PQ₂, PQ₂₂₃ and purified fractions from CVT-E002, PQ₂ and PQ₂₂₃- In another embodiment, the fraction is CVT-E002.

In yet another embodiment, the present invention is directed to a kit for treating an infant subject to cause elevated levels of NK cells in the subject when an adult, comprising, in separate containers, (1) at least one ginseng fraction and (2) a pharmaceutically acceptable carrier or diluent therefor. In another embodiment, the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng. In another embodiment, the fraction is a fraction of Panax quinquefolius. In another embodiment, the fraction is selected from the group consisting of CVT-E002, PQ₂, PQ₂₂₃ and purified fractions from CVT-E002, PQ₂ and PQ₂₂₃. In another embodiment, the fraction is CVT-E002.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a graph showing the body weights at euthanasia of control and ginseng fraction CVT-E002—treated mice killed between 0 and 5 days (age 21-26 days), or 7-8 weeks of age.

FIG. 2 is a graph showing the effect of ginseng fraction CVT-E002 on hemopoietic and immune cell populations in the spleen of mice killed between 0 and 5 days (age 21-26 days).

FIG. 3 is a graph showing the effect of ginseng fraction CVT-E002 on hemopoietic and immune cell populations in the bone marrow of mice killed between 0 and 5 days (age 21-26 days).

FIG. 4 is a graph showing the effect of ginseng fraction CVT-E002 on hemopoietic and immune cell populations in the spleen of mice killed between 7-8 weeks of age.

FIG. 5 is a graph showing the effect of ginseng fraction CVT-E002 on hemopoietic and immune cell populations in the bone marrow of mice killed between 7-8 weeks of age.

### DETAILED DESCRIPTION OF PREFERRED EMBODIMENTS

When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

As used herein and in the claims, the terms and phrases set out below have the meanings which follow.

“Active ingredient” means any ginseng fraction or component thereof capable of modifying or modulating the function of at least one given biological system.

“Preventing” is meant to refer to a process by which a hematological malignancy is obstructed or delayed.

“Reducing” is meant to refer to a process by which a hematological malignancy is lessened or diminished.

“Protection” or “protective immunity” means the ability of a ginseng fraction to protect (partially or totally) against a hematological malignancy.

“Hematological malignancy” means an abnormal proliferation of blood cells, or a disease of the lymph nodes and multiple myeloma. The abnormal proliferation of blood cells is acute lymphocytic leukemia, acute myelogenous leukemia, chronic lymphocytic leukemia including the subtype hairy cell leukemia or chronic myelogenous leukemia.

“Biocompatible” means generating no significant undesirable host response for the intended utility. Most preferably, biocompatible materials are non-toxic for the intended utility. Thus, for human utility, biocompatible is most preferably non-toxic to humans or human tissues.

“Carrier” means a suitable vehicle which is biocompatible and pharmaceutically acceptable, including for instance, one or more solid, semisolid or liquid diluents including food items, excipients, adjuvants, flavours, or encapsulating substances which are suitable for administration.

“Subject” means humans or other vertebrates. The subject may be a child or an adult.

A “functional food” is similar in appearance to, or may be, a conventional food that is consumed as part of a usual diet, and is demonstrated to have physiological benefits and/or reduce the risk of disease beyond basic nutritional functions, i.e. they contain an active ingredient.

A “nutraceutical” is a product isolated or purified from foods that is generally sold in medicinal forms not usually associated with foods. A nutraceutical is should have a physiological benefit or provide protection against disease.

“Effective amount” denotes any amount of a formulation of a ginseng fraction which will prevent or reduce hematological malignancy upon administration. The amount of ginseng fraction administered will vary with the age and type of host, and the type and concentration of the formulation being applied. Appropriate amounts in any given instance will be readily apparent to those skilled in the art or capable of determination by routine experimentation.

“Pharmaceutically acceptable” denotes a substance which does not significantly interfere with the effectiveness or the biological activity of the ginseng fraction and which has an acceptable toxic profile for the host to which it is administered.

A “fraction” is meant to refer to a concentrated preparation obtained from extraction of a plant or plant part with a suitable solvent such as, for example, water, ethanol, a mixture thereof, oils or any other suitable solvent well known in the state of the art of plant extraction. The fraction or extract can be used as such if pharmacologically acceptable (i.e., having pharmacological activity for the intended purpose), or the solvent of the resulting solutions is removed and the residue is used as such or after further work up, for example, after resolving or re-suspending in a suitable solvent. The term “plant” is understood to mean the whole plant and plant parts comprising the active ingredients, for example, the leaves, the stems, the fruits or roots.

“Ginseng” is meant to refer to any variety and type of ginseng including, but not limited to, those listed below.

TABLE 1
Varieties and Types of Ginseng

| Latin name(s) | Common name(s) |
|---|---|
| Panax quinquefolius | North American/Canadian |
| Panax trifolia | Eastern region of North America |
| Panax ginseng | Asian ginseng |
| Panax japonicus | Korean ginseng |
| Panax schinseng | Oriental ginseng |
| Panax notoginseng | Japanese ginseng |
| Panax pseudoginseng | Chinese ginseng |
| Panax vietnamensis | Nepalese ginseng |
| Panax elegatior | Vietnamese ginseng |
| Panax wangianus | Wild ginseng |
| Panax bipinratifidus | Green or fresh ginseng |
| | Red ginseng |
| | White ginseng |
| | Xi Yang Shen |
| | Ren Shen/Gao Li Shen |
| | Tienchi/Sanchi |
| | Sâm Ng [symbol] c Linh |

It will be understood by those skilled in the art that there are many other genus of Panax genus plants belonging to Araliaceae which may be used within the context of the present invention. The term “ginseng” also includes wild or processed ginseng. Wild ginseng is ginseng which has not been planted and cultivated domestically, but grows naturally and is harvested from wherever it is found to be growing. Processed ginseng includes, for example, fresh or green ginseng, white ginseng, and red ginseng. Fresh or green ginseng is raw ginseng harvested in the field. White ginseng is obtained by drying fresh ginseng, and red ginseng is obtained by steaming fresh ginseng followed by drying the steamed ginseng.

A “ginseng fraction” or “ginseng fractions” is meant to refer to fractions made from any variety and type of ginseng as listed in Table 1 or described above, and subfractions obtained from these ginseng fractions, which exhibit the activity of preventing or reducing hematological malignancies, or elevating NK cells, as verified by conducting one or more in vitro or in vivo pharmacological evaluations.

“CVT-E002” is meant to refer to an exemplary ginseng fraction obtained from Panax quinquefolius, and which has immunoregulating properties (as previously described in U.S. Pat. Nos. 6,432,454; 7,067,160; 7,186,423 and 7,413,756 which are hereby incorporated by reference). CVT-E002 exhibits the additional activity of preventing hematological malignancies as described herein.

“PQ₂” is meant to refer to an exemplary ginseng fraction obtained from Panax quinquefolius, and which has immunoregulating properties as previously described in U.S. Pat. Nos. 6,432,454; 7,067,160; 7,186,423 and 7,413,756 which are hereby incorporated by reference.

“PQ₂₂₃” is meant to refer to an exemplary ginseng fraction obtained from Panax quinquefolius, and which has immunoregulating properties as previously described in U.S. Pat. Nos. 6,432,454; 7,067,160; 7,186,423 and 7,413,756 which are hereby incorporated by reference.

“Infant” is meant to refer to a person from birth through to development of a fully matured immune system.

It will be appreciated by those skilled in the art that fractions from plants or plant parts other than ginseng, or synthetic fractions which may equally well be used in the present context, are within the scope of the present invention, as long as their chemical properties and activities are sufficiently similar to the ginseng fraction used herein.

The present invention is directed to a fraction of ginseng, a pharmaceutical composition comprising the fraction in combination with another medicament or with one or more pharmaceutically acceptable carriers including food items, for the prevention or reduction of a hematological malignancy, or elevation of NK cells. In one embodiment, the ginseng fraction is a fraction of Panax quinquefolius. In another embodiment, the ginseng fraction is selected from the group consisting of CVT-E002, PQ₂, PQ₂₂₃ and purified fractions from CVT-E002, PQ₂ and PQ₂₂₃- In a further embodiment, the ginseng fraction is CVT-E002.

Preferably, the condition characterized by a hematological malignancy is selected from the group consisting of abnormal proliferation of blood cells, a disease of the lymph nodes and multiple myeloma. Even more preferably, the abnormal proliferation of blood cells is selected from the group consisting of acute lymphocytic leukemia, acute myelogenous leukemia, chronic lymphocytic leukemia including the subtype hairy cell leukemia and chronic myelogenous leukemia. In one embodiment, the hematological malignancy is leukemia.

Further, the present invention is directed to use of a ginseng fraction for the preparation of a pharmaceutical composition or a food item for the prevention or reduction of a hematological malignancy, or elevation of NK cells. In one embodiment, the ginseng fraction is a fraction of Panax quinquefolius. In another embodiment, the ginseng fraction is selected from the group consisting of CVT-E002, PQ₂, PQ₂₂₃ and purified fractions from CVT-E002, PQ₂ and PQ₂₂₃. In another embodiment, the ginseng fraction is CVT-E002. In a further embodiment, the hematological malignancy is leukemia.

Further, the present invention is directed to a method of preventing or reducing a hematological malignancy in a subject, when the subject is an infant, by administering to the subject an effective amount of a fraction made from ginseng, a pharmaceutical composition comprising the fraction in combination with another medicament or with one or more pharmaceutically acceptable carriers including food items. In one embodiment, the ginseng fraction is a fraction of Panax quinquefolius. In another embodiment, the ginseng fraction is selected from the group consisting of CVT-E002, PQ₂, PQ₂₂₃ and purified fractions from CVT-E002, PQ₂ and PQ₂₂₃. In another embodiment, the ginseng fraction is CVT-E002. In another embodiment, the hematological malignancy is leukemia.

Further, the present invention is directed to a method of producing elevated NK cells in an adult subject, comprising administering to the subject when the subject is an infant an effective amount of a ginseng fraction.

Further, the present invention is directed to a method of producing elevated NK cells in an adult subject, comprising administering to the subject when the subject is an infant an effective amount of a ginseng fraction without the requirement for further administration of the ginseng fraction when the subject is an adult.

Further, the present invention is directed to a method of increasing NK cells in an infant, comprising administering to an infant an effective amount of a ginseng fraction.

Further, the present invention is directed to a kit for treating an infant subject to cause elevated levels of NK cells in the subject when an adult, comprising, in separate containers, (1) at least one ginseng fraction and (2) a pharmaceutically acceptable carrier or diluent therefor.

Further, the present invention is directed to a kit for treating an infant subject to cause elevated levels of NK cells in the subject when an adult, comprising, in separate containers, (1) at least one ginseng fraction and (2) a pharmaceutically acceptable carrier or diluent therefor without the requirement for further administration of the ginseng fraction when the subject is an adult.

The ginseng fraction of this invention is effective in the prevention or reduction of a hematological malignancy such as leukemia in an animal model system, or elevation of NK cells. Additionally, since the ginseng fraction is prepared from a natural, edible product, the potential for side effects is decreased. As demonstrated using a murine model system in Example 1, the ginseng fraction of this invention confers a protective immunity against leukemia during infancy. NK cells have a short life span of less than two days, and are replaced rapidly and exponentially in the bone marrow, from which they are exported, never to return (Miller, 1982; Koo and Mankay, 1986; Pollack and Rosse, 1987; Kalland, 1986). It is possible to significantly augment the proliferation and absolute number of NK cells in the bone marrow and spleen of infant mice when the ginseng fraction is administered daily during their pre-weaning period. Surprisingly, this protective immunity is maintained in adulthood after withdrawal of the ginseng fraction. The production and absolute numbers of NK cells remained significantly elevated compared to control adults injected with only the PBS vehicle during pre-weaning.

The ginseng fraction appears to have a distinctly positive influence on the developing (infant) bone marrow microenvironment (stromal cells), the latter, in adults at least, having been shown to govern the production of NK cells (Dussault and Miller, 1993, 1995; Miller, 1982, 1984). The exemplary ginseng fraction CVT-E002 appears to imbue permanent, positive changes in the NK cell-generating stromal cells. CVT-E002 is known to stimulate the same cytokines which have been demonstrated by others to augment the bone marrow stromal cell development and function.

NK cells in the adult mouse and human can be stimulated numerically and/or functionally by certain cytokines, i.e., IL-2 (Koo and Manyak, 1986; Keever et al., 1990; Fuchshuber and Lotzova, 1991; Vecchini et al., 1993; Tsuji and Pollack, 1995). Production or generation of new NK cells is controlled by the stromal cells of the bone marrow microenvironment (Tsuji and Pollack, 1995; Pollack and Rosse, 1987; Kalland, 1986; Van den Brink et al., 1990; Rosmaraki et al., 2001; Colucci et al., 2003). In the infant, NK cells are produced in the bone marrow at the same levels, and have the same antitumoricidal, functional potency as the adult; however, they are actively suppressed in vivo in the infant (Dussault and Miller, 1993, 1995). Stromal cells themselves are also under regulatory influences, i.e., soluble factors such as IL-1, IL-6 and TNF-α (Sensebe et al., 1995; Kuznetsov et al., 1997; Yan et al, 1990; Gronthos and Simmons, 1995; Andrades et al., 1999; Satomura et al., 1998), and these factors can be significantly augmented by the exogenous administration of the ginseng fraction (Wang et al, 2002, 2004). If indeed, these cytokines, and/or other soluble agents which drive stromal cell development and function, were increased in the infant in vivo by daily administration of the ginseng fraction, then it is reasonable to infer that the mechanism by which the ginseng fraction increases NK cells, is an indirect one, acting by stimulating cytokines which then create a supernormal stromal cell (microenvironment) network - the latter in turn governing the development of supernormal levels of NK cells. Still further support for this possibility is the demonstration that in the primitive, developing bone marrow, the stromal cells are proliferating, while the stromal cells of the adult do not (Bianco et al., 1999, 2000). Hence, any positive qualitative and/or quantitative influence which the ginseng fraction has had on the stromal cells in the infant has been sustained into adulthood, where they, in turn, would account for the elevated levels of NK cells.

The same explanation applies to the observed increase in absolute numbers in the mature and proliferating precursor members of the granulocyte series of cells, which are also (like NK cells) generated in the bone marrow, and exported from that organ to the spleen, and also have a short life span. The stromal cell microenvironment of the bone marrow also produces soluble factors which drive granulocytopoiesis (Sensebe et al., 1997; Mohr et al., 1997; Hubin et al, 2005). The role of granulocytes in tumor combat has been well documented (Di Carlo et al., 2001; Giovarelli et al., 2000; Kindzelski and Petty, 1999; Otten et al., 2005; Okrent et al., 1990).

Infant mammals, containing a significantly super-normal population of NK cells, may be adequately fortified to combat infant/juvenile cancers which may arise during the immediate post-weaning period.

The ginseng fraction may thus be considered as a “dietary vaccine.” The significance of exposing mammalian infants to the ginseng fraction is the apparent maintenance of this elevated level of immunity into adulthood, after the ginseng fraction has been withdrawn. Provision of the ginseng fraction to increase immunity in young animals/humans could have profound prophylactic value. Several food staples consumed by children—for example milk and bread—are currently treated with assorted vitamins and minerals, to imbue health benefits which may not otherwise be forthcoming.

The ginseng fraction is typically prepared by first drying and powderizing the ginseng plant or plant parts and then performing an extraction process using an appropriate solvent, typically water, ethanol, ethanol/water mixture, methanol, butanol, iso-butanol, acetone, hexane, petroleum ether or other organic solvents. The fraction or extract may then be further evaporated and thus concentrated to yield a dried extract by means of spray drying, vacuum oven drying, or freeze-drying. Processes for making exemplary ginseng fractions selected from the group consisting of CVT-E002, PQ₂, PQ₂₂₃ and purified fractions from CVT-E002, PQ₂ and PQ₂₂₃ from a water soluble extract of the root portion of Panax quinquefolius have previously been described in U.S. Pat. Nos. 6,432,454; 7,067,160; 7,186,423 and 7,413,756 which are hereby incorporated by reference.

Once prepared, the ginseng fraction is evaluated to assess and confirm the activity of preventing or reducing hematological malignancies, or elevating NK cells by conducting one or more in vitro or in vivo pharmacological evaluations. In the present invention, such evaluations include, but are not limited to, an in vivo study of the effects of an exemplary ginseng fraction, CVT-E002, on hemopoietic and immune cell populations (see Example). For the present invention, any pharmacological evaluations are suitable, provided that they are focused upon indication of the above activities in either the ginseng fraction, a representative sample from a batch of the ginseng fraction in the event of large scale manufacturing, or a subfraction of the ginseng fraction. Batch-to-batch quality of the product may be certified by ChemBioPrint™ Technology, which assures its chemical as well as pharmacological consistency, as described in U.S. Pat. No. 6,156,291 which is hereby incorporated by reference.

Various formulations of the ginseng fraction can be prepared for administration to a subject such as, for example, oral dosage forms, topical preparations, or injectable preparations.

Powders of the ginseng fraction may be used in that form directly as a loose powder or encapsulated powder. Alternatively, powders may be formulated into capsules, soft capsules, caplets, tablets, pills, lozenges, granulate and similar dosage forms. Further, powders may be formulated within liquid pervious membranes such as filters, meshes, sachets and the like, such as a tea bag-type infuser, for generating liquids containing the dissolved ginseng fraction.

The powder form of the ginseng fraction may be incorporated into liquids, formulated as solutions, dispersions or suspensions by dissolving the ginseng fraction, for example as an aqueous medicine, a syrup, drink, tincture, or drop. The ginseng fraction may be administered alone, or with a carrier such as saline solution, an alcohol or water. An effective daily amount of the ginseng fraction will vary with the subject, but will be less than is toxic while still providing effect.

The ginseng fraction may be incorporated into topical preparations such as, for example, creams, ointments, lotions, gels, balms, patches, pastes, spray solutions, aerosols and the like.

The ginseng fraction may be incorporated into injectable preparations such as, for example, solutions, suspensions, emulsions and the like.

Pharmaceutical compositions may be prepared to include the ginseng fraction as an active ingredient, in combination with another medicament or with one or more biocompatible or non-toxic, pharmaceutically acceptable carriers, diluents and excipients, as are well known, see for example, U.S. Pat. Nos. 6,432,454; 7,067,160; 7,186,423 and 7,413,756 which are hereby incorporated by reference; and Hardman and Limbird (eds) (2001) Goodman and Gilman's: The Pharmacological Basis of Therapeutics, 10^{th} Ed., McGraw-Hill Professional. For standard dosages of conventional pharmacological agents, see for example, Physician's Desk Reference, 62 Ed., (2008); and U.S. Pharmacopeia—National Formulary (2008). Compositions may also include flavors, colorings, coatings, etc. All agents must be non-toxic and pharmaceutically acceptable for the intended purpose, and must not substantially interfere with the activity of the ginseng fraction.

Formulations of the ginseng fraction may lose some activity with aging and are thus either prepared in stable forms, or prepared fresh for administration, for example in multicomponent kit form so as to avoid aging and to maximize the effectiveness of the ginseng fraction. Suitable kits or containers are well known for maintaining the phases of formulations separate until the time of use. For instance, a kit containing the ginseng fraction in powder form may be packaged separately from a sterile carrier such as saline solution, alcohol or water, and possibly other ingredients in dosage specific amounts for mixing at the time of use. The ginseng fraction may be provided in a “tea bag”-type infuser, pouch or sachet, for generating liquid formulations at the time of use. The tea bag-type infuser is advantageous in that the pouch may serve as a filter for small particulates of the powder that may be detrimental with certain types of administration (for example, via injection or infusion). Particulates may also be removed by for example, filtration.

The dosage of the ginseng fraction depends upon many factors that are well known to those skilled in the art, for example, the particular form of the ginseng fraction; the age, weight and general health condition of the subject; any concurrent therapeutic treatments; and the experience and judgment of the clinician or practitioner administering the ginseng fraction. However, suitable daily dosage may be found in the range 0.5 to 5000 mg/kg body weight. A preferable range for a suitable daily dosage of the ginseng fraction is within the range of 1 to 4800 mg/kg body weight. Even more preferably, the suitable ginseng fraction daily dosage is within the range of about 3 to 1600 mg/kg body weight. The suitable dose should be administered in the range of 1 to 10 individual doses. Preferably, the suitable dose should be 1 to 5 individual doses. Even more preferably, the individual dose should be 2 to 4 individual doses.

The ginseng fraction, pharmaceutical compositions and food items comprising the ginseng fraction can be administered to a subject by various routes. All modes of administration are contemplated, for example, orally, via injection or infusion, intraperitoneally, topically, nasally, ocularly, vaginally or rectally, in solid, semi-solid or liquid dosage forms as appropriate and in unit dosage forms suitable for easy administration of fixed dosages. For oral administration, the ginseng fraction may be taken without (i.e., on an empty stomach) or with liquid or food. There is nothing critical about the concentration of the fraction in the composition as long as the composition can be administered to a subject.

In the above description and following examples it is to be understood that the mention of the preferred embodiment of CVT-E002 is exemplary only and that the described utility in activities would be appropriate to all ginseng fractions able to produce the desired activity.

The invention will now be further elucidated by the following Example.

### EXAMPLE

### Effect of CVT-E002 on Hemopoietic and Immune Cell Populations

The effect of CVT-E002, a proprietary extract of North American ginseng, Panax quinquefolius comprising unique polysaccharides (poly-furanosyl-pyranosyl-saccharides) of CV Technologies, Inc., Edmonton, AB, Canada, was examined in vivo on the hemopoietic and immune cells of infant (pre-weaned) mice.

Animals:

Pregnant CD-1 mice (Charles River Laboratories, St. Constant, QC, Canada) were housed upon arrival one/cage and maintained under pathogen-free conditions (micro-isolator cages) in a temperature/humidity regulated room with a twelve hour day/night cycle, in the Animal Care Facility of McGill University. Animals were provided water and food ad libidum and the date of birth of each litter was recorded. Pups were not manipulated until seven days of age, at which time the experiments were initiated. Regular assessments of sentinel mice contained in the room consistently demonstrated absence of all common mouse pathogens.

Administration of CVT-E002:

CVT-E002 was administered via intraperitoneal (i.p.) injections daily for fourteen days, beginning at seven days of age, irrespective of gender. The extract in powdered form was suspended in sterile phosphate buffered saline (PBS) pH 7.2. Twenty mg of CVT-E002 were administered to seven day old infants (body weight: 6.5 gm) in a volume of 50 μl PBS. As the infants grew with age, the dose of CVT-E002 was adjusted upward accordingly. Control infants received the PBS vehicle only. Each morning, experimental (CVT-E002-injected) and control (vehicle-injected) infants were weighed and the values recorded. The dose administered to the seven day old infants was one which was “downsized” by body weight, from a most potent dose in adult mice—most potent being defined as that which, when administered to adult mice bearing a virus-induced leukemia, produced a significantly high survival rate.

Preparation of Free Cell Suspensions of Bone Marrow and Spleen:

Mice were killed by CO₂ asphyxiation at 21-26 days of age (0-5 days after terminating CVT-E002) and at 7-8 weeks of age. Single cell suspensions of the bone marrow and spleen were prepared by standard laboratory methods. Both femurs (bone marrow source) and the spleen of each animal were aseptically removed and transferred to ice cold Minimal Essential Medium (MEM) (GIBCO Invitrogen Corp. Burlington, ON, Canada), containing 10% heat-inactivated (56° C., 30 min) fetal bovine serum (FBS) (GIBCO Invitrogen Corp., Burlington, ON, Canada). Spleens were pressed through a stainless steel screen mesh into medium, and bone marrow was removed by repeated flushing of the femurs with medium. Free cell suspensions from both organs were obtained by gentle repeating pipetting. Suspensions were then layered for 7 min onto 1.5 ml newborn calf serum (NCS) (GIBCO Invitrogen Corp., Burlington, ON, Canada), to allow the sedimentation of any non-cellular debris into the NCS. The aggregate-free supernatants were removed and centrifuged for 7 min (1100 rpm, 4° C.), and the resulting pellet was re-suspended in a fixed volume of fresh medium. The total number of nucleated cells, as well as the viability (Trypan Blue exclusion test; 0.04% dye in PBS: pH 7.2) (GIBCO Invitrogen Corp., Burlington, ON, Canada) was obtained by means of a hemocytometer (American Optical Co., Buffalo, NY, USA). Free cell suspensions of the bone marrow and spleen were adjusted to a final concentration of 40×10⁶ cells/ml PBS.

Immunolabelling of NK Cells:

Mature NK cells, all of which bear the surface molecule, ASGM-1 (asialogangliotetrasyliramide) were processed for microscopic visualization by an indirect immunoperoxidase method. Using 96 multi-well plates (Sarstedt Inc., Montreal, QC, Canada), 100 μl of suspension (bone marrow or spleen) was incubated with 100 μl of primary antibody-rabbit anti-ASGM-1 (Wako Pure Chemicals, Dallas, Tex., USA) at a dilution of 1:40 in medium for 30 min on ice. After incubation, the cells were centrifuged for 7 min (1100 rpm, 4° C.), followed by two consecutive washes with 100 μl of medium and centrifuged as above. After the final wash, the pellets were re-suspended and incubated with 100 μl of the secondary biotinylated antibody-anti-rabbit IgG (Sigma-Aldrich, Oakville, ON, Canada) at a concentration of 1:100 in medium for 30 min on ice. Cell suspensions were centrifuged and washed twice as above, before being re-suspended in 4.5 ml of cytospotting medium (0.009% NaCl, 0.001% EDTA and 0.05% bovine serum albumin in distilled water, pH 7.4) (Sigma-Aldrich, Oakville, ON, Canada). The cells were then cytocentrifuged (5 min, 1000 g) onto Superfrost Plus™ microscope slides and rapidly air-dried to avoid cell shrinkage. All slides were fixed in pure methanol for 30 min on ice, re-hydrated progressively with PBS pH 7.2 (25%, 50%, 75% and 100%) for 5 min each, then bathed for 10 min in 3% hydrogen peroxide solution (Fisher Scientific Inc., Ottawa, ON, Canada) to block endogenous peroxidase activity. Slides were washed for 10 min in PBS and incubated with 100 μl of avidin-biotin horseradish peroxidase complex (ABC) solution (Dako Diagnostics Inc., Mississauga, ON, Canada) for 45 min in a fully humidified chamber. Thereafter, the slides were washed as above in PBS to remove any residual ABC solution before being immersed in a 3-3′diaminobenzidine solution (0.125 gm DAB, 66.6 μl 30% H₂O₂ in 250 ml PBS at pH 7.6) for 13 min followed by two consecutive 10 min washes in PBS. All cytocentrifuged cells (“cytospots”) were subsequently stained with MacNeal's tetrachrome hematologic stain (Sigma-Aldrich, Oakville, ON, Canada), mounted with Cytoseal 60™ (Richard-Allan Scientific, Inc., Kalamazzo, Mich., USA) and coverslipped. This double staining method (immunolabelling and tetrachrome) readily permits the identification of all hemopoietic and immune cell lineages as well as NK cells (uniquely immunolabelled).

Differential Analysis of Hemopoietic and Immune Cells in the Bone Marrow and Spleen:

For both the bone marrow and the spleen, mature granulocytes, precursor granulocytes, nucleated erythroid cells (precursors to the blood-borne red blood cells responsible for gas exchange), NK cells, other lymphocytes (T, B), and monocytes, were identified by methods according to Brousseau and Miller (2005); Whyte and Miller (1998); Miller and Kearney (1997); Mahoney et al. (1998); and Sun et al. (1999).

Briefly, differential cell counts were recorded, using light microscopy (1000 spleen cells/cytospot/mouse and 2000 bone marrow cells/cytospot/mouse) for every experimental (CVT-E002 injected) and control (vehicle-injected) mouse at both time intervals (21-26 days; 7-8 weeks). For each organ, the percentages of each cell lineage were recorded. The absolute numbers of individual cell lineages were then obtained by converting these percentage values, via the known total organ cellularity recorded from the hemocytometer at the time of organ extraction.

Statistical Analysis:

The influence of treatment on all cell lineages in both organs for CVT E002 versus control mice was determined by the Student t-test (two-tailed). The differences between the means of experimental and control values for each cell lineage for each organ were compared and the probability values calculated. Values of p<0.05 were considered statistically significant.

Results:

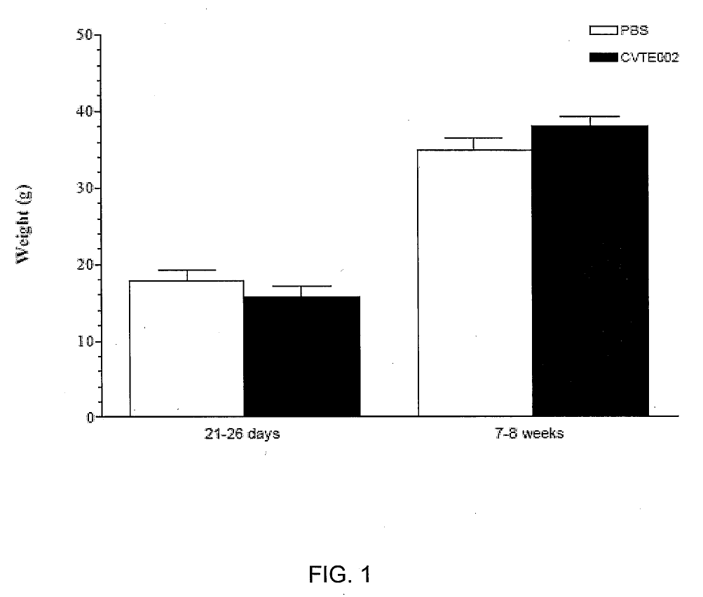

Administration of CVT-E002 for 14 days (7-21 days of age) produced no significant differences in body weight when assessed at sampling (euthanasia) time (21-26 days or 7-8 weeks of age) (FIG. 1, mean±s.e. N=5-6 mice/histogram (age 21-26 days); N=4-5 mice/histogram (7-8 weeks of age)).

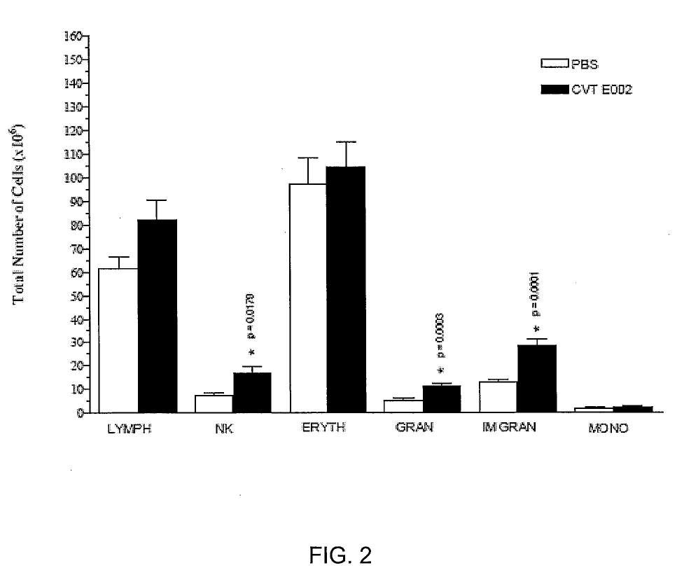

Within the first five days after terminating CVT-E002, natural killer cells (NK) were significantly elevated in absolute numbers in the infant spleen versus control (FIG. 2, mean±s.e. N=5-6 mice/histogram (age 21-26 days); N=4-5 mice/histogram (7-8 weeks of age); LYMPH—T and B lymphocytes; ERYTH—nucleated, organ-based precursors of blood-borne red blood cells; MONO—monocytes, necessary accessory cells in the immune system). Both precursor (IM GRAN) and mature, lytic granulocytes (GRAN) were also significantly increased in absolute numbers.

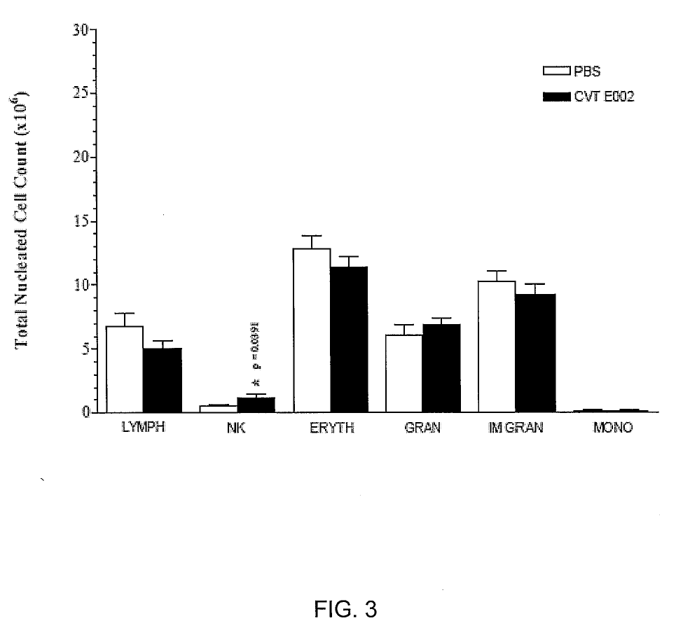

The bone marrow of these mice (during the first 5 days after terminating CVT-E002), also contained significantly elevated absolute numbers of natural killer (NK) cells (FIG. 3). This is an especially important observation since NK cells, once generated in their bone marrow birth site, never re-circulate back into it. Consequently, any elevation in NK cell numbers in the bone marrow necessarily means that CVT-E002 has stimulated new NK cell production/proliferation in the infant (mean±s.e., 5-6 mice per histogram).

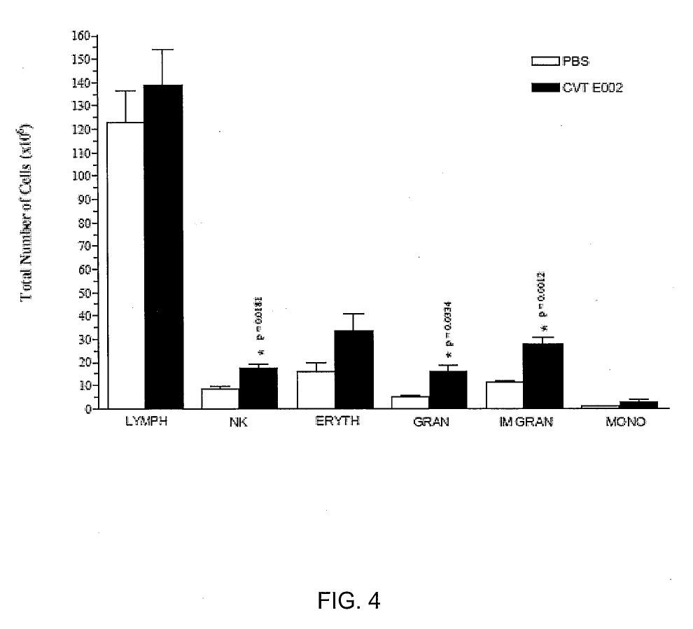

In 7-8 week old (adult) mice exposed as infants to CVT-E002, there was a significant elevation in absolute numbers, in the spleen, both of NK cells and of precursors and mature forms of granulocytes (FIG. 4, mean±s.e., 4-5 mice per histogram).

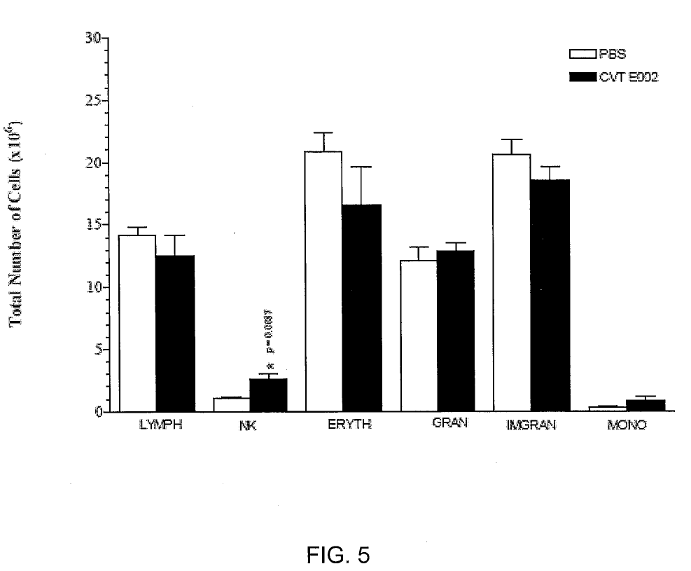

The absolute numbers of NK cells, in the bone marrow of these adult mice, are also statistically elevated, indicating a sustained, super-normal production of these cells in spite of the fact that the stimulating agent (CVT-E002) was withdrawn at weaning (FIG. 5, mean±s.e., 4-5 mice per histogram). By contrast, the absolute numbers of cells in all other hemopoietic and immune cell populations in both the spleen and bone marrow, were not influenced by the ginseng extract either immediately after CVT-E002 withdrawal (age 21-26 days) (FIGS. 2 and 3), or several weeks after withdrawal (age 7-8 weeks) (FIGS. 4 and 5).

### REFERENCES

The following references are incorporated herein by reference as if reproduced in their entirety. All references are indicative of the level of skill of those skilled in the art to which this invention pertains.
Andrades, J. A., B. Han, J. Becerra et al. (1999) A recombinant human TGF-beta 1 fusion protein with collagen-binding domain promotes migration, growth, and differentiation of bone marrow mesenchymal cells. Exp Cell Res. 250:485-498.
Argyris, B. F. (1968) Role of macrophages in immunological maturation. J Exp Med. 128:459-467.
Argyris, B. F. (1978) Suppressor activity in the spleen of neonatal mice. Cell Immunol. 36:354-362.
Argyris, B. F. (1981) Suppressor factor produced by neonatal mouse spleen cells. Cell Immunol. 62:412-424.
Bianco, P., M. Riminucci, S. Kuznetsov et al. (1999) Multipotential cells in the bone marrow stroma: regulation in the context of organ physiology. Crit Rev Eukaryot Gene Expr. 9:159-173.
Bianco, P. and P. G. Robey (2000) Marrow stromal stem cells. J Clin Invest. 105(12):1663-1668.
Biron, C. A. and R. M. Welsh (1982) Activation and role of natural killer cells in virus infections. Med Microbiol Immunol (Berl) 170:155-172.
Biron, C. A. and R. M. Welsh (1982) Blastogenesis of natural killer cells during viral infection in vivo. J Immunol 129:2788-2795.
Brousseau, M. and S. C. Miller (2005) Enhancement of natural killer cells and increased survival of aging mice fed daily Echinacea root extract from youth. Biogerontology 6:157-163.
Calkins, C. E. and O. Stutman (1978) Changes in suppressor mechanisms during post-natal development in mice. J Exp Med. 147:87-97.
Chiscon, M. Q. and E. S. Golub (1972) Functional development of the interacting cells of the immune response. I. Development of T cell and B cell function. J Immunol. 108:1379-1386.
Colucci, F., M. A. Caligiuri and J. P. Di Santo (2003) What does it take to make a natural killer? Nature Rev (Immunology) 3:413-425.
Di Carlo, E., G. Forni, P. L. Lollini et al. (2001) The intriguing role of polymorphonuclear neutrophils in antitumor reactions. Blood 97(2):339-345.
Dussault, I. and S. C. Miller (1995) Suppression of natural killer cell activity in infant mice occurs after target cell binding. Nat Immun. 14:35-43.
Dussault, I. and S. C. Miller (1993) Stimulation of natural killer cell numbers but not function in leukemic infant mice: A system primed in infancy allows survival in adulthood. Nat Immun. 12:66-78.
Fuchshuber, P. R. and E. Lotzova (1991) Feeder cells enhance oncoloytic and proliferative activity of long-term human bone marrow interleukin-2 cultures and induce different lymphocyte subsets. Cancer Immunol Immunother. 33:15-20.
Giovarelli, M., P. Cappello, G. Forni et al. (2000) Tumor rejection and immune memory elicited by locally released IEC chemokine are associated with an impressive recruitment of antigen-presenting cells, lymphocytes and granulocytes. J Immunol. 164:3200-3206.
Gronthos, S. and P. J. Simmons (1995) The growth factor requirements of STRO-1-positive human bone marrow stromal precursors under serum-deprived conditions in vitro. Blood 85:929-940.
Herberman, R. B., M. E. Nunn, and D. H. Larvin (1975) Natural cytotoxic reactivity of mouse lymphoid cells against syngeneic and allogeneic tumors. I. Distribution of reactivity and specificity. Int J Cancer 16:216-229.
Hertell-Wulff, B. and S. Strober (1988) Immunosuppressive lymphokines derived from natural suppressor cells. J Immunol. 240:2633-2638.
Holan, V., M. Lipoldova and A. Zajicova (1991) Immunological nonreactivity of newborn mice: Immaturity of T cells and selective action of neonatal suppressor cells. Cell Immunol. 137:216-223.
Hubin, F., C. Humblet, Z. Belaid et al. (2005) Murine bone marrow stromal cells sustain in vivo the survival of hematopoietic stem cells and the granulopoietic differentiation of more mature progenitors. Stem Cells 23(10):1626-1633.
Kalland, T. (1986) Generation of natural killer cells from bone marrow precursors in vitro. Immunology 57:493-498.
Keever, C. A., K. Pekle, M. V. Gazzola et al. (1990) NK and LAK activities from human marrow progenitors: I. The effects of interleukin-2 and interleukin-1. Cell Immunol. 126:211-226.
Keissling, R., E. Klein, H. Pross et al. (1975) Natural killer cells in the mouse. II Cytotoxic cells with specificity for mouse Maloney leukemia cells. Characteristics of the killer cell. Eur J Immunol. 5:117-121.
Kindzelski, A. L. and H. R. Petty (1999) Early membrane rupture events during neutrophil-mediated antibody-dependent tumor cell cytolysis. J Immunol. 162:3188-3192.
Klinman, N. R. (1976) The acquisition of B cell competence and diversity. Am J Pathol. 85:693-703.
Koo, G. C. and C. L. Manyak (1986) Generation of cytotoxic cells from murine bone marrow by human recombinant IL2. J Immunol. 137:1751.
Kuznetsov, S. A., A. J. Friedenstein and P. G. Robey (1997) Factors required for bone marrow stromal fibroblast colony formation in vitro. Br J Haematol. 97:561-570.
Lala, P. K., V. Santar, H. Libenson et al. (1985) Changes in host natural killer regulation in mice during tumor development. I. Kinetic and in vivo significance. Cell Immunol. 93:250-264.
Landahl, C. A. (1976) Ontogeny of adherent cells. I. Distribution and ontogeny of cells participating in the response to sheep erythrocytes in vitro. Eur J Immunol. 6:130-134.
Mahoney, M. X., N. L. Currier and S. C. Miller (1998) Natural killer cell levels in older adult mice are gender-dependent: thyroxin is a gender-independent natural killer cell stimulant. Nat Immun. 16:165-174.
Maier, T., J. H. Holda and H. N. Claman (1989) Murine natural suppressor cells in the newborn, in the bone marrow and after cyclophosphamide. Genetic variations and dependence on IFN-gamma. J Immunol. 143:491-498.
Miller, S. C., M. T. Gallagher, S. K. Datta et al (1981) Development of natural killer cell activity and genetic resistance to bone marrow transplantation with age: Effect of neonatal thymectomy. Scand J Jmmunol. 13:105- 110.
Miller, S. C. (1982) Production and renewal of murine natural killer cells in the spleen and bone marrow. J Immunol. 129(5):2282-2286.
Miller, S. C. (1984) Life history of cells mediating natural resistance to tumor cells and bone marrow transplants: The respective roles of cell lineage commitment and host environment in determining strain characteristics of natural resistance to foreign marrow grafts. Am J Anat. 170:367-376.
Miller, S. C. and S. L. Kearney (1997) Effect of in vitro administration of transretinoic acid on the hemopoietic cell populations of spleen and bone marrow: profound strain differences between A/J and C57B1/6J mice. Lab An Sci. 48(1):74-80.
Mohr, A. M., J. S. Upperman, R. Taneja et al. (1997) Differential effects of acute hypoxia and endotoxin on the secretion and expression of bone marrow interleukin-1 and interleukin-6. Shock 7(5):324-331.
Mosier, D. E. and B. M. Johnson. (1975) Ontogeny of mouse lymphocyte function. II. Development of the ability to produce antibody is modulated by T lymphocytes. J Exp Med. 141: 216-226.
Okrent, D. G., A. K. Lichtenstein and T. Ganz. (1990) Direct cytotoxicity of polymorphonuclear leukocyte granule proteins to human lung-derived cells and endothelial cells. Am Rev Respir Dis 141:179-185.
Otten, M. A., E. Rudolph, M. Dechant et al. (2005) Immature neutrophils mediate tumor cell killing via IgA but not IgG Fc receptors. J Immunol. 174(9):5472-5480.
Pollack, S. B. and C. Rosse. (1987) The primary role of murine bone marrow in the production of natural killer cells: A cytokinetic study. J Immunol. 139:2149-2156.
Riccardi, C., T. Barlozzari, A. Santoni et al. (1981) Transfer to cyclophosphamide-treated mice of natural killer (NK) cells and in vivo natural reactivity against tumors. J Immunol. 126:1284-1289.
Rosmaraki, E. E., I. Douagi, C.Roth et al. (2001) Identification of committed NK cell progenitors in adult murine bone marrow. Eur J Immunol. 31(6): 1900-1909.
Satomura, K., A. R. Derubeis, N. S. Fedarko et al. (1998) Receptor tyrosine kinase expression in human bone marrow stromal cells. J Cell Physiol. 177:426-328.
Sensebe, L., J. Li, M.Lilly et al. (1995) Nontransformed colony-derived stromal cell lines from normal human marrows. I. Growth requirement and myelopoiesis supportive ability. Exp Hematol. 23:507-513.
Sensebe, L., B. T. Mortensen, P. Fixe et al. (1997) Cytokines active on granulomonopoiesis: release and consumption by human marrow myeloid stromal cells. Br J Haematol. 98:274-282.
Sherwin, W. K. and D. T. Rowlands. (1975) Determinants of the hierarchy of humoral immune responsiveness during ontogeny. J Immunol. 115:1549-1554.
Spear, P. G., A. L. Wang, U. Rutishauser, et al. (1973) Characteristics of splenic lymphoid cells in fetal and newborn mice. J Exp Med. 138:557-573.
Spear, P. G. and G. Edelman. (1974) Maturation of the humoral immune response in mice. J Exp Med. 139:249-263.
Strober, S. (1984) Natural suppressor (NS) cells, neonatal tolerance and total lymphoid irradiation: exploring obscure relationships. Ann Rev Immunol. 2:219-237.
Sun, L. Z-Y., N. L. Currier and S. C. Miller. (1999) The American Coneflower: A prophylactic role involving nonspecific immunity. J Alt Comp Med. 5(5):437-446.
Tsuji, J. M. and S. B. Pollack (1995) Maturation of murine natural killer precursor cells in the absence of exogenous cytokines requires contact with bone marrow stroma. Nat Immun. 14:44-56.
Van den Brink, M. R. M., S. S. Boggs, R. B. Herberman et al. (1990) The generation of natural killer (NK) cells from NK precursor cells in rat longterm bone marrow cultures. J Exp Med. 172:303-313.
Vecchini, F., D. Delfino, K. D. Patrene et al (1993) Generation of natural killer cells from long-term cultures of mouse bone marrow. Nat Immun. 12:1-16.
Velardi, A. and M. D. Cooper (1984) An immunofluorescence analysis of the ontogeny of myeloid, T, and B lineage cells in the mouse hemopoietic tissues. J Immunol. 133:672-677.
Wang, M., L. J. Guilbert, L. Ling et al. (2002) Immunomodulating activity of CVT-E002, a proprietary extract from North American ginseng (Panax Quinquefolium). J Pharm Pharmacol. 22:1515-1523.
Wang, M., L. J. Guilbert, J. Li et al. (2004) A proprietary extract from North American ginseng (Panax quinquefolium) enhances IL-2 and IFN-γ production in murine spleen cells induced by Con-A. Int Immunopharm. 4:311-315.
Whyte, A. L. and S. C. Miller (1998) Strain difference in natural killer cell mediated immunity among mice: a possible mechanism for the low natural killer cell activity of A/J mice. Immunobiology 199:23-38.
Yan, Z-J., Q-R. Wang, I. K. McNiece et al (1990) Dissecting the hematopoietic microenvironment. VII. The production of an autostimulatory factor as well as a CSF by unstimulated murine marrow fibroblasts. Exp Hematol. 18:348-354.

As will be apparent to those skilled in the art, various modifications, adaptations and variations of the foregoing specific disclosure can be made without departing from the scope of the invention claimed herein.

What is claimed is:

1. A fraction of ginseng for the prevention or reduction of hematological malignancies, or for elevation of NK cells.

2. The fraction of claim 1, wherein the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng.

3. The fraction of claim 2, wherein the fraction is a fraction of Panax quinquefolius.

4. The fraction of claim 3, wherein the fraction is selected from the group consisting of CVT-E002, PQ₂, PQ₂₂₃ and purified fractions from CVT-E002, PQ₂ and PQ₂₂₃.

5. The fraction of claim 4, wherein the fraction is CVT-E002.

6. A pharmaceutical composition comprising the fraction of claim 1 in combination with another medicament or with one or more pharmaceutically acceptable carriers including food items for the prevention or reduction of hematological malignancies, or elevation of NK cells.

7. The composition of claim 6, wherein the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng.

8. The composition of claim 7, wherein the fraction is a fraction of Panax quinquefolius.

9. The composition of claim 8, wherein the fraction is selected from the group consisting of CVT-E002, PQ₂, PQ₂₂₃ and purified fractions from CVT-E002, PQ₂ and PQ₂₂₃.

10. The composition of claim 9, wherein the fraction is CVT-E002.

11. A method of preventing or reducing hematological malignancies comprising administering to a subject, when the subject is an infant, an effective amount of a ginseng fraction, a pharmaceutical composition comprising the fraction in combination with another medicament or with one or more pharmaceutically acceptable carriers including food items.

12. The method of claim 11, wherein the hematological malignancy is selected from the group consisting of an abnormal proliferation of blood cells, a disease of the lymph nodes and multiple myeloma.

13. The method of claim 12, wherein the blood cells are leukocytes and/or erythrocyte precursors.

14. The method of claim 12, wherein the abnormal proliferation of blood cells is selected from the group consisting of acute lymphocytic leukemia, acute myelogenous leukemia, chronic lymphocytic leukemia including the subtype hairy cell leukemia, and chronic myelogenous leukemia.

15. The method of claim 14, wherein said acute myelogenous leukemia is erythroleukemia.

16. The method of claim 11, wherein the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng.

17. The method of claim 16, wherein the fraction is a fraction of Panax quinquefolius.

18. The method of claim 17, wherein the fraction is selected from the group consisting of CVT-E002, PQ₂, PQ₂₂₃ and purified fractions from CVT-E002, PQ₂ and PQ₂₂₃.

19. The method of claim 18, wherein the fraction is CVT-E002.

20. A method of preventing leukemia in a subject comprising administering to a subject an effective amount of a ginseng fraction, a pharmaceutical composition comprising the fraction in combination with another medicament or with one or more pharmaceutically acceptable carriers including food items.

21. The method of claim 20, wherein the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng.

22. The method of claim 21, wherein the fraction is a fraction of Panax quinquefolius.

23. The method of claim 22, wherein the fraction is selected from the group consisting of CVT-E002, PQ₂, PQ₂₂₃ and purified fractions from CVT-E002, PQ₂ and PQ₂₂₃.

24. The method of claim 23, wherein the fraction is CVT-E002.

25. The method of claim 20, wherein the subject is a child.

26. A method of producing elevated NK cells in an adult subject, the method comprising administering to the subject when the subject is an infant an effective amount of a ginseng fraction.

27. The method of claim 26, wherein the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng.

28. The method of claim 27, wherein the fraction is a fraction of Panax quinquefolius.

29. The method of claim 28, wherein the fraction is selected from the group consisting of CVT-E002, PQ₂, PQ₂₂₃ and purified fractions from CVT-E002, PQ₂ and PQ₂₂₃.

30. The method of claim 29, wherein the fraction is CVT-E002.

31. A method of increasing NK cells in an infant, comprising administering to an infant an effective amount of a ginseng fraction.

32. The method of claim 31, wherein the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng.

33. The method of claim 32, wherein the fraction is a fraction of Panax quinquefolius.

34. The method of claim 33, wherein the fraction is selected from the group consisting of CVT-E002, PQ₂, PQ₂₂₃ and purified fractions from CVT-E002, PQ₂ and PQ₂₂₃.

35. The method of claim 34, wherein the fraction is CVT-E002.

36. A kit for treating an infant subject to cause elevated levels of NK cells in the subject when an adult, comprising, in separate containers, (1) at least one ginseng fraction and (2) a pharmaceutically acceptable carrier or diluent therefor.

37. The kit of claim 36, wherein the fraction is made from a ginseng selected from the group consisting of Panax quinquefolius, Panax trifolia, Panax ginseng, Panax japonicus, Panax schinseng, Panax notoginseng, Panax pseudoginseng, Panax vietnamensis, Panax elegatior, Panax wangianus, Panax bipinratifidus, green or fresh ginseng, white ginseng, and red ginseng.

38. The kit of claim 37, wherein the fraction is a fraction of Panax quinquefolius.

39. The kit of claim 38, wherein the fraction is selected from the group consisting of CVT-E002, PQ₂, PQ₂₂₃ and purified fractions from CVT-E002, PQ₂ and PQ₂₂₃.

40. The kit of claim 39, wherein the fraction is CVT-E002.
